(19) United States
(12) Patent Application Publication
(10) Pub. No.: US 2006/0084639 A1
(43) Pub. Date: Apr. 20, 2006

(54) Compositions containing piperacillin and tazobactam useful for injection

(75) Inventors: Jonathan Marc Cohen, Monroe, NY (US); Syed M. Shah, East Hanover, NJ (US); Mahdi Fawzi, Morristown, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/250,716

(22) Filed: Oct. 14, 2005

Related U.S. Application Data
(60) Provisional application No. 60/719,177, filed on Sep. 22, 2005.
(60) Provisional application No. 60/618,872, filed on Oct. 14, 2004.

(51) Int. Cl. A61K 31/43; A61K 31/407; A61K 31/198

(52) U.S. Cl. 514/192; 514/210.09; 514/561

(57) ABSTRACT

The invention provides a pharmaceutical composition comprising piperacillin, tazobactam, an aminocarboxylic acid, and a buffer in a sodium lactate diluent. The invention further relates to a method of treating a bacterial infection and an LR condition in a human which comprises administering to said human an effective amount of a pharmaceutical composition comprising piperacillin, tazobactam, an aminocarboxylic acid, and a buffer in a sodium lactate diluent.

This application claims priority from copending provisional application Ser. No. 60/618,872 filed Oct. 14, 2004 and Ser. No. 60/719,177 filed Sep. 22, 2005 the entire disclosures of which is hereby incorporated by reference.

### FIELD OF THE INVENTION

The invention relates to a pharmaceutical composition comprising piperacillin, tazobactam, an aminocarboxylic acid, and a buffer in a sodium lactate diluent. The invention further relates to a method of treating a bacterial infection and an LR condition in a human which comprises administering to said human an effective amount of a pharmaceutical composition comprising piperacillin, tazobactam, an aminocarboxylic acid, and a buffer in a sodium lactate diluent.

### BACKGROUND OF THE INVENTION

Zosyn® is an antibiotic marketed product containing piperacillin sodium and tazobactam sodium. As listed on the label, Zosyn® is incompatible with lactated Ringer's solution.

There is a need for a pharmaceutical composition which overcomes the incompatibility of Zosyn® with lactated Ringer's solution.

### BRIEF SUMMARY OF THE INVENTION

The invention provides a pharmaceutical composition comprising piperacillin, tazobactam, an aminocarboxylic acid and a buffer in a sodium lactate diluent.

The invention further provides a method of treating a bacterial infection and an LR condition in a human which comprises administering to said human an effective amount of a pharmaceutical composition comprising piperacillin, tazobactam, an aminocarboxylic acid and a buffer in a sodium lactate diluent.

In some embodiments of the invention, an aminocarboxylic acid is preferably EDTA. In some embodiments of the invention, the buffer is citric acid, preferably sodium citrate.

In further embodiments of the invention, the sodium lactate diluent is lactated Ringer's solution.

In some embodiments of the invention, the sodium lactate diluent is Hartmann's solution.

### DETAILED DESCRIPTION OF THE INVENTION

The pharmaceutical compositions of the invention have the advantage over marketed pharmaceutical compositions of piperacillin-tazobactam wherein a solution for infusion of piperacillin-tazobactam in a sodium lactate solution, in particular lactated Ringer's solution or Hartmann's solution demonstrate compatibility by having particulate counts not more than 6000 particles ≧10 μm and not more than 600 particles ≧25 μm and a chemical potency greater than 90% of the initial concentration. Compatibility for example with lactated Ringer's solution may be achieved by buffering the pharmaceutical compositions of the invention with a buffer, for example, citrate to maintain the suitable pH range of about 6.0 to about 7.5 in the presence of an aminocarboxylic acid or a salt thereof. A preferred pH is about 6.5. In an embodiment of the invention the aminocarboxylic acid is EDTA in the form of edetate disodium dihydrate. Optionally the aminocarboxylic acid may be added in a hospital setting before administration to a patient or may also be premixed in a ready-to-use pharmaceutical composition.

In particular, in a hospital setting, the compositions of piperacillin-tazobactam of the invention in the presence of a buffer and an aminocarboxylic acid may be advantageously added to a human patient via a “Y” site connection on an intravenous line. A “Y” site connection is a common practice which permits additional medicaments to be added while a therapeutic infusion is in progress.

The following definitions are used throughout the application.

“LR condition” means any condition which calls for the use of a sodium lactate diluent which include lactated Ringer's solution or Hartmann's solution or other similar sodium lactate solution. Typical non-limiting LR conditions include burns, replacement of fluid deficits, trauma, blood substitutes, haemorrhage, infections and the like. Optionally, Hartmann's solution may replace lactated Ringer's.

“Treating” refers to reversing, alleviation of symptoms or inhibiting the progress of a bacterial infection.

“Diluent” means the fluid for administration to a patient, such as via parenteral (e.g., subcutaneous, intravenous, bolus injection, intramuscular, or intraarterial) administration. In particular diluents are a sodium lactate diluent more preferably lactated Ringer's solution or Hartmann's solution. Typically, the sodium lactate diluent is added by intravenous infusion. Typically lactated Ringer's solution is used in the United States, and Hartmann's solution is used in Europe.

“Administering” means a treatment process wherein an effective amount of a pharmaceutical composition of the invention is delivered to a human patient.

“Bacterial infection” is the proliferation of a bacteria pathogen caused by Gram-positive and/or Gram-negative bacteria.

“Effective amount” is an amount of a pharmaceutical composition of the invention, where upon administration is capable of reducing or preventing the proliferation of bacteria or reducing the symptoms of the bacterial infection.

“HPLC” means high pressure liquid chromatography.

The term, “aminocarboxylic acid” preferably includes: ethylenediaminetetraacetic acid (EDTA) and salts thereof, for example, ethylenediaminetetraacetic acid, calcium disodium salt (preferably as the hydrate), dicalcium EDTA; ethylenediaminetetraacetic acid, diammonium salt (preferably as the hydrate); ethylenediaminetetraacetic acid, dipotassium salt (preferably as the dihydrate); ethylenediaminetetraacetic acid, disodium salt (preferably as the dihydrate and, if desired, as the anhydrous form); ethylenediaminetetraacetic acid, tetrasodium salt (preferably as the hydrate); ethylenediaminetetraacetic acid, tripotassium salt (preferably as the dihydrate); ethylenediaminetetraacetic acid, trisodium salt (preferably as the hydrate) and ethylenediaminetetraacetic acid disodium salt, USP (preferably as the dihydrate). Other “aminocarboxylic acids” include: for example, diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N, N′,N′-tetraacetic acid (EGTA), trans-1,2-diaminocyclohexane-N,N,N′,N′-tetraacetic acid (CyDTA) or a pharmaceutically acceptable salt thereof (normally as a sodium salt).

“Aminoglycoside antibiotics” are selected from amikacin and tobramycin.

The terms Compound Sodium Lactate Infusion, European Lactated Ringer's Solution, and Hartman's Solution are used interchangeably.

| | | Lactated |
|---|---|---|
| | Compound Sodium Lactate | Ringer's Injection |
| Ingredient | Infusion BP (% w/v) | USP (% w/v) |
| Sodium | 0.27-0.32 | 0.285-0.315 |
| Potassium | 0.019-0.022 | 0.0142-0.0173 |
| Calcium Chloride | 0.025-0.029 | 0.018-0.022 |
| dihydrate | | |
| Lactate | 0.23-0.28 | 0.231-0.261 |
| Total Chloride | 0.37-0.42 | 0.368-0.408 |
| pH | 5.0-7.0 | 6.0-7.5 |

Piperacillin sodium is the preferred form of piperacillin in the compositions of the present invention. Piperacillin free acid is a possible source of piperacillin for use in making the compositions of the present invention. The free acid may be converted to the sodium salt during the formulation process. Piperacillin sodium is derived from D(−)-α-aminobenzylpenicillin. The chemical name of piperacillin sodium is sodium (2S,5R,6R)-6-[(R)-2-(4-ethyl-2,3-dioxo-1-piperazinecarboxamido)-2-phenylacetamido]-5 3,3-dimethyl-7-oxo-4-thia-1-azabicyclo(3.2.0) heptane-2-carboxylate, with a chemical formula of C₂₃H₂₆N₅O₇SNa and a molecular weight of 539.6.
Tazobactam sodium is the preferred form of tazobactam in the compositions of the present invention. Tazobactam free acid is a possible source of tazobactam for use in making the compositions of the present invention. The free acid may be converted to the sodium salt during the process of forming the pharmaceutical compositions of the invention. Tazobactam sodium, a derivative of the penicillin nucleus, is a penicillanic acid sulfone. Its chemical name is sodium (2S,3S,5R)-3-methyl-7-oxo-3-(1H-1,2,3-triazol-1-ylmethyl)-4-thia-1-azabicyclo-(3,2,0)heptane-2-carboxylate-4, 4-dioxide. The chemical formula for tazobactam sodium is C₁₀H₁₁N₄NaO₅S and the molecular weight is 322.3.
The pharmaceutical compositions of the invention may be buffered with citrate or other suitable buffers to maintain the pH within the preferred range of about 6.0 to about 7.5. Citrate is the preferred buffer because it can maintain the pH of the solution without significant drug degradation. The addition of a buffer is desired for controlling the pH to enhance stability. Preferably, a suitable amount of sodium citrate used to buffer the formulation, controls the pH for maximum stability without significantly catalyzing or degrading the drug, or causing pain to the patient upon infusion. Sodium citrate dihydrate is the preferred form for the buffer used in the present invention. As used herein citrate is citric acid or salts thereof, preferably sodium citrate. Sodium citrate is available as trisodium citrate anhydrous, trisodium citrate dihydrate, and trisodium citrate pentahydrate. Sodium citrate dihydrate is also known as trisodium citrate dihydrate and is preferred. The preferred form is trisodium citrate dehydrate. There are also several hydration forms of monobasic and dibasic sodium citrate which may replace the trisodium citrate, in whole or in-part.
Typical pharmaceutical compositions of the invention include the following ranges: Piperacillin in the range of about 8 mg/ml to about 500 mg/ml; more preferably about 12 mg/ml to about 300 mg/ml;
Tazobactam in the range of about 0.1 mg/ml to about 125 mg/ml; more preferably about 1.5 mg/ml to about 75 mg/ml;
Citrate in the range of about 0.25 mg/ml to about 25 mg/ml; more preferably about 0.6 mg/ml to about 15 mg/ml;
An aminocarboxylic acid in the range of about 0.002 mg/ml to about 10 mg/ml; more preferably about 0.003 to about 1 mg/ml;
Optionally added to pharmaceutical compositions of the invention is dextrose in the range of about 5 mg/ml to about 100 mg/ml.
Optionally added to pharmaceutical compositions of the invention are aminoglycosides in the range of about 0.1 mg/ml to about 75 mg/ml.
Experimental Methods

### Determination of Piperacillin, and Tazobactam in Typical Pharmaceutical Compositions of the Invention

A typical pharmaceutical composition of the invention, having 3000 mg/vial of piperacillin, 375 mg of Tazobactam, 150 mg of citrate and 0.75 mg of EDTA was reconstituted with 15 ml of sterile water for injection. The resulting solution was transferred to a flexible container containing 250 ml of Lactated Ringer's Solution, USP (United States Pharmacopeia). The container was mixed with gentle inversions forming the drug solution and sampled immediately for chemical analysis. The container was then stored at ambient temperature for 24 hours and re-sampled. This test was performed in duplicate.

The chemical analysis was performed according to the following analytical method. The analytical procedure is summarized as follows:

1) 4 ml aliquots of the drug solution is drawn and transferred to a 250 ml volumetric flask.
2) The flask is filled, qs. to 250 ml with dilution solvent (25% acetonitrile/75% water, v/v)
3) Mixing is performed using gentle inversions of the flask.
4) Aliquots are then taken for HPLC analysis.
5) The method further uses an isocratic flow of a mobile phase containing 25% acetonitrile/75% water, v/v.
6) The recommended column is a Phenomenex, Luna 3 micron Pheny-Hexyl 130×4.6 mm.
7) The UV absorbance detector is set at 210 nM.
8) Adjustments were made in calculations to compensate for volume overages in the Lactated Ringer's container (268 ml were found to be present).

A summary of the HPLC analysis data is provided as Table I.

TABLE I

| Typical | | | | Total |
|---|---|---|---|---|
| pharmaceutical | | Piperacillin | Tazobactam | Unidentified |
| composition | Time Hr | (% LC) | (% LC) | Degradants |
| Sample 1^{a} | 0 | 119.1 | 115.0 | 0.1% |
| Sample 1 | 24 | 100.8 | 95.2 | 0.1% |
| Sample 2 | 0 | 100.2 | 96.8 | 0.1% |
| Sample 2 | 24 | 101.3 | 95.7 | 0.1% |

^{a}An investigation concluded that the cause of the higher than expected piperacillin and tazobactam result was due to a pipetting error.

A summary of the HPLC analysis data performed on a piperacillin/tazobactam formula without a buffer or an aminocarboxylic acid using the analytical procedure for Samples 1 and 2 is provided in Table II.

TABLE II

| Typical | | | | Total |
|---|---|---|---|---|
| pharmaceutical | | Piperacillin | Tazobactam | Unidentified |
| composition | Time Hr | (% LC) | (% LC) | Degradants |
| Sample 3 | 0 | 101.9 | 100.3 | 1.8% |
| Sample 3 | 24 | 94.7 | 97.6 | 6.4% |
| Sample 4 | 0 | 98.9 | 100.4 | 2.0% |
| Sample 4 | 24 | 96.4 | 98.5 | 6.1% |

The compatibility of piperacillin-tazobactam vial products containing an aminocarboxylic acid and a buffer was evaluated in the presence of the compound sodium lactate to determine compatibility up to 24 hours at room temperature storage. Admixture samples were also tested for compatibility after being stored for 1 week under refrigerated (2-8° C.) conditions. For these admixtures, the concentration tested was based on the commercially available volume (250 ml) of lactated Ringer's solution.
Additional testing was performed which confirmed that admixture solutions prepared using reconstituted piperacillin-tazobactam vial products containing an aminocarboxylic acid and a buffer that had been stored for not less than 48 hours at refrigerated (2-8° C.) conditions in the vial prior to admixing, demonstrated equivalent compatibility in this diluent.
Using the above test procedures, samples of each piperacillin-tazobactam vial products containing an aminocarboxylic acid and a buffer concentration were tested immediately (T=0 hour) after being admixed into the diluent. Remaining samples are stored at ambient laboratory conditions (about 20° C.) and tested again 24 hours (T=24 hour) later. A set of samples were tested after 1 week under refrigerated (2-8° C.) conditions. At all time points, the tests conducted included visual appearance and description, using the procedures described in USP <788>/Ph. Eur. Chapter 2.9.19 Particulate Matter (Light Obscuration Particle Counts), and by HPLC chemical potency for Piperacillin and Tazobactam.
All samples tested were determined to be clear and free of particulates by visual inspection. All particulate counts, were less than the USP/Ph. Eur. acceptance criteria of not more than 6000 particles ≧10 μm and not more than 600 particles ≧25 μm. As determined by HPLC, chemical potency was greater than 90% of the initial concentration.
The data in these studies demonstrated that piperacillin-tazobactam vial products containing an aminocarboxylic acid and a buffer are compatible with compound sodium lactate, for up to 24 hours when stored at room temperature for up to 1 week, when stored at refrigerated conditions (2-8° C.) and that admixture dilutions may be prepared with reconstituted product stored at refrigerated conditions (2-8° C.) in the vial for up to 48 hours.
Several standard tests were performed to evaluate the chemical and physical compatibility of piperacillin-tazobactam vial products containing an aminocarboxylic acid and a buffer with a sodium lactate diluent. The first test was conducted to determine if piperacillin-tazobactam vial products containing an aminocarboxylic acid and a buffer are compatible in admixture with a sodium lactate diluent for up to 24 hours at room temperature storage. Admixtures were also tested after being held for 1 week under refrigerated (2-8° C.) conditions. High (16 mg/ml, piperacillin; 2 mg/ml, tazobactam) and low (8 mg/ml, piperacillin; 1 mg/ml, tazobactam) concentrations of piperacillin-tazobactam vial products containing an aminocarboxylic acid and a buffer were prepared as admixtures with this diluent. They were based on a 250 mL bag.
In another standard test, a sodium lactate diluent was evaluated for compatibility with piperacillin-tazobactam vial products containing an aminocarboxylic acid and a buffer as admixtures that were prepared using reconstituted drug product that had been stored for not less than 48 hours at refrigerated (2-8° C.) conditions prior to admixing.
These admixtures were tested using the high and low concentrations described above.
The following batches of piperacillin-tazobactam vial products containing an aminocarboxylic acid and a buffer were used in the compatibility test procedures:
a. 4.5 g/vial, Batch A9MY/1, manufactured by Wyeth.
b. 2.25 g/vial, Batch A9N3/1, manufactured by Wyeth.
   The sodium lactate diluent used was Compound Sodium Lactate infusion, 500 mL containers, Lot 05E10D, manufactured by MacoPharma, Expiry Date May 2007
   Vial Reconstitution and Admixture Preparation Compound Sodium Lactate
   Each piperacillin-tazobactam vial products containing an aminocarboxylic acid and a buffer were reconstituted at 5 ml of diluent per gram of piperacillin.
   A sodium lactate was used to both reconstitute vials and subsequently prepare the admixture solutions.
   The admixtures were prepared to represent two vial strengths, lowest and highest piperacillin-tazobactam vial products containing an aminocarboxylic acid and a buffer vial strengths (2.25 g and 4.5 g, respectively). The resultant admixture concentrations evaluated are tabulated in Table III.
   For a sodium lactate diluent, a 250 mL volume was used to prepare both the lowest and highest admixture concentration.
   Admixture Testing Procedure
   In this study, samples of each piperacillin-tazobactam vial products containing an aminocarboxylic acid and a buffer concentration were tested immediately (T=0 hour) after being admixed into the diluent lactated Ringer's solution. Remaining samples were stored at ambient laboratory conditions (about 20° C.) and tested at and 24 hours (T=24 hour). A set of admixture samples were also tested after being stored under refrigerated (2-8° C.) conditions for 1 week.
   Analytical Test Methods
   The following test methods were used to analyze the samples:
   HPLC Assay for Piperacillin and Tazobactam
   Visual Observation for Appearance and Description
   USP <788>/Ph. Eur. Chapter 2.9.19 Particulate Matter (Light Obscuration Method for sub-visible particulate counts)
   Acceptance Criteria
   All samples were visually clear and compliant with current USP/Ph. Eur. specification for subvisible particulates at T=0 hour, T=24 hours, and T=1 week under refrigerated (2-8° C.) conditions (if applicable). For HPLC Assay, all samples after T=24 hours (or T=1 week under refrigerated (2-8° C.) conditions) were not less than 90% of the initial (T=0 hour) concentration for both piperacillin and tazobactam.
   Particulate counts did not change as a function of time. The potencies were not substantially changed. There were no changes seen in any of product attributes over a 24 hour period.
   Appearance and Description
   In the studies, the samples tested were determined to be clear and free of particulates by visual inspection. There were no differences observed in the diluent with piperacillin-tazobactam vial products containing an aminocarboxylic acid and a buffer over a 24 hour admixture testing period.
   Sub-Visible Particulate Matter
   All sub-visible particulate counts were acceptable, indicating no significant formation of particulates. All particulate counts, both at T=0 hour and T=24 hours were well under the USP/Ph. Eur. acceptance criteria of less than 6000 particles ≧10 μm and less than 600 particles ≧25 μm. There were no differences observed in the various diluents with piperacillin-tazobactam vial products containing an aminocarboxylic acid and a buffer over the 24-hour admixture testing period. Results are presented in Table IV.
   Potency
   In the tests, all HPLC chemical potency data for Piperacillin and Tazobactam met the acceptance criteria of not less than 90% of the initial concentration and remained unchanged over the 24-hour admixture testing period.

Acceptable physical stability (i.e. Appearance and Description and Sub-Visible Particulates) and potency stability for admixtures prepared in the diluents, when stored at ambient conditions (about 20° C.) for up to 24 hours after admixture preparation:

Sodium Lactate Intravenous Infusion

Acceptable physical stability (i.e. Appearance and Description and Sub-Visible Particulates) and potency stability for admixtures prepared in this same diluent, but with reconstituted product stored at refrigerated conditions (2-8° C.) for up to 48 hours. In addition, these admixtures demonstrated compatibility when stored at ambient conditions (about 20° C.) for up to 24 hours after admixture preparation.

TABLE III
Admixture Concentrations Used To Determine Compatibility of Piperacillin-
Tazobactam Vial Products Containing An Aminocarboxylic acid and a Buffer With
Lactated Ringer's Intravenous Diluents

| | | | piperacillin-tazobactam | |
|---|---|---|---|---|
| | | | vial products containing an | |
| | | | aminocarboxylic acid and a | |
| Compatibility Study | Reconstitution | | buffer Admixture | |
| Name | Diluent | Admixture Diluent | Concentration | |
| Lactated Ringer's | Lactated Ringer's | Lactated Ringer's | LOW | 8 mg/mL piperacillin; |
| Preliminary Study | Injection-USP | Injection-USP | | 1 mg/mL tazobactam |
| | | | HIGH | 16 mg/mL piperacillin; |
| | | | | 2 mg/mL tazobactam |

TABLE IV

| piperacillin- | | | | | | |
|---|---|---|---|---|---|---|
| tazobactam | | | | | | |
| vial products | | | | | | |
| containing | | | | | | |
| an aminocarboxylic acid | USP/Ph. Eur | | | | | |
| and a buffer | Particulate Counts | | | | HPLC Potency Assay | |
| Admixture | 10 μm¹ | | 25 μm² | | Piperacillin³ | Tazobactam³ |
| Concentration | t = 0 | t = 24 | t = 0 | T = 24 | t = 24 | t = 24 |
| LOW | 1947 | 1057 | 37 | 3 | | |
| 8 mg/mL piperacillin; | 970 | 1407 | 40 | 23 | 100.1 | 99.8 |
| 1 mg/mL tazobactam | 1243 | 873 | 17 | 20 | | |
| HIGH | 1107 | 2083 | 17 | 87 | | |
| 16 mg/mL piperacillin; | 3217 | 2550 | 153 | 97 | 100.4 | 100.1 |
| 2 mg/mL tazobactam | 3260 | 1770 | 70 | 47 | | |

¹Not more than 6000
²Not more than 600
³Not less than 90% initial

Further Studies
Additional studies to evaluate the chemical and physical compatibility of admixtures of piperacillin-tazobactam vial products containing an aminocarboxylic acid and a buffer with the intravenous fluid Lactated Ringer's Injection-USP were performed.
The compatibility of piperacillin-tazobactam vial products containing an aminocarboxylic acid and a buffer vial products was evaluated with Lactated Ringer's Injection-USP to determine if such admixture solutions are compatible up to 24 hours at room temperature storage. For Lactated Ringer's Injection, the concentration tested was based on the available volume (250 ml) of this solution that most closely matched the volumes described in the current commercial product (50-150 ml).
An additional study was performed which confirmed that admixture solutions prepared using reconstituted drug product that had been stored for not less than 48 hours at refrigerated (2-8° C.) conditions in the vial prior to admixing, demonstrated equivalent compatibility the diluent.
Samples of each piperacillin-tazobactam vial products containing an aminocarboxylic acid and a buffer were tested immediately (T=0 hour) after being admixed into the diluent. Remaining samples are stored at ambient laboratory conditions (about 20° C.) and tested again 24 hours (T=24 hour) later. In the preliminary study, samples were also tested after 4 hours (T=4 hour) storage at ambient laboratory conditions (about 20° C.). At all time points, the tests conducted included visual appearance and description, USP <788>/Ph. Eur. Chapter 2.9.19 Particulate Matter (Light Obscuration Particle Counts), and HPLC chemical potency for Piperacillin and Tazobactam.
In the studies, all samples tested were determined to be clear and free of particulates by visual inspection. All particulate counts, were well under the USP/Ph. Eur. acceptance criteria of not more than 6000 particles ≧10 μm and not more than 600 particles ≧95 μm. Likewise, all HPLC chemical potency data met the acceptance criteria of not less than 90% of the initial concentration.
The data in these studies demonstrated that piperacillin-tazobactam vial products containing an aminocarboxylic acid and a buffer is compatible admixture with lactated Ringers solution tested for up to 24 hour when stored at room temperature, that admixture dilutions may be prepared with reconstituted product stored at refrigerated conditions (2-8° C.) in the vial for up to 48 hours, and that reconstitution of the piperacillin-tazobactam vial products containing an aminocarboxylic acid and a buffer lyophilized dosage form with several preserved diluents had no impact on the compatibility and stability of the subsequent admixture prepared with saline.
Lactated Ringer's Study
High and low concentrations of piperacillin-tazobactam vial products containing an aminocarboxylic acid and a buffer were prepared as admixtures in Lactated Ringer's diluent.
The compatibility up to 24 hours at room temperature storage of piperacillin-tazobactam vial products containing an aminocarboxylic acid and a buffer in Lactated Ringer's Injection-USP was determined. High and low concentrations of piperacillin-tazobactam vial products containing an aminocarboxylic acid and a buffer were prepared as admixtures in the diluent and the Lactated Ringer's Injection, is based on a 250 mL bag.
Reconstituted Product Hold Time Prior to Admixture Study
In this study, the diluents were evaluated for compatibility with piperacillin-tazobactam vial products containing an aminocarboxylic acid and a buffer and were also prepared in highest and lowest admixture concentrations. However, in this study, the admixtures were prepared using reconstituted drug product that had been stored for not less than 48 hours at refrigerated (2-8° C.) conditions prior to admixing.
Materials and Methods

### Materials

The following batches of piperacillin-tazobactam vial products containing an aminocarboxylic acid and a buffer were used in the compatibility studies:

a. 4.5 g/vial, Batch A93374, manufactured by Wyeth, on 8 Sep. 2004.
b. 2.25 g/vial, Batch A87605, manufactured by Wyeth, on 6 Jul. 2004.
c. 40.5 g/vial, Batch A98715, manufactured by Wyeth, on 17 Sep. 2004.
   The following lots of intravenous diluents were used in one or more of the compatibility studies described.
Lactated Ringer's Injection, USP, 250 mL containers, Lot J4J577, manufactured by B. Braun, Expiry Date 01/06

### Methods

Vial Reconstitution and Admixture Preparation

Each piperacillin-tazobactam vial products containing an aminocarboxylic acid and a buffer were reconstituted at 5 ml of diluent per gram of piperacillin.

The admixtures for the studies were prepared with diluent to represent the lowest and highest piperacillin-tazobactam vial products containing an aminocarboxylic acid and a buffer vial strengths (2.25 g and 4.5 g, respectively). The resultant admixture concentrations evaluated in each of the studies are tabulated in Tables V-X.

For Lactated Ringers Injection, a 250 mL volume was used to prepare both the lowest and highest admixture concentration, as this is the smallest volume container available.

For Lactated Ringer's Injection-USP, low and high concentrations were prepared based on the lowest and highest piperacillin-tazobactam vial strengths (respectively) dissolved in 250 mL of this diluent.

Admixture Testing Procedure

In the Lactated Ringer's Preliminary Study, samples of each piperacillin-tazobactam vial products containing an aminocarboxylic acid and a buffer concentration were tested immediately (T=0 hour) after being admixed into the diluent. Remaining samples were stored at ambient laboratory conditions (about 20° C.) and tested at 4 hours (T=4 hour) and 24 hours (T=24 hour).

Analytical Test Methods

The following test methods were used to analyze the samples in these compatibility studies:

HPLC Assay for Piperacillin and Tazobactam

Visual Observation for Appearance and Description

USP <788>/Ph. Eur. Chapter 2.9.19 Particulate Matter (Light Obscuration Method for sub-visible particulate counts)

Acceptance Criteria

All samples should be visually clear and compliant with current USP/Ph. Eur. specification for subvisible particulates at T=0 hour, T=4 hours (if applicable) and T=24 hours. For HPLC Assay, all samples after T=24 hours should not be not less than 90% of the initial (T=0 hour) concentration for both piperacillin and tazobactam.

Results

The USP <788>/Ph. Eur. Chapter 2.9.19 and HPLC Assay data obtained for each diluent in each of the studies are provided in Tables V to X. Acceptance criteria were met in all studies. Particulate counts did not change as a function of time in each solution in the chart. The potencies were not substantially changed.

Discussion

There were no changes seen in any of product attributes over a 24 hour period. The results of these studies indicate acceptable stability.

Appearance and Description

In all four studies, the samples tested were determined to be clear and free of particulates by visual inspection. There were no differences observed in the diluent with piperacillin-tazobactam vial products containing an aminocarboxylic acid and a buffer over a 24 hour admixture testing period.

Sub-Visible Particulate Matter

In all four studies, all sub-visible particulate counts were acceptable, indicating no significant formation of particulates. All particulate counts, both at T=0 hour, T=4 hours (where applicable) and T=24 hours were well under the USP/Ph. Eur. acceptance criteria of not more than 6000 particles ≧10 μm and not more than 600 particles ≧25 μm. There were no differences observed in the various diluents with piperacillin-tazobactam vial products containing an aminocarboxylic acid and a buffer over the 24-hour admixture testing period.

Potency

In the studies, all HPLC chemical potency data for Piperacillin and Tazobactam met the acceptance criteria of not less than 90% of the initial concentration and remained unchanged over the 24-hour admixture testing period.

Acceptable physical stability (i.e. Appearance and Description and Sub-Visible Particulates) and potency stability for admixtures prepared in the diluent Lactated Ringer's Injection, USP, when stored at ambient conditions (about 20° C.) for up to 24 hours after admixture preparation.

Acceptable physical stability (i.e. Appearance and Description and Sub-Visible Particulates) and potency stability for admixtures prepared in these same diluent, but with reconstituted product stored at refrigerated conditions (2-8° C.) for up to 48 hours. In addition, these admixtures demonstrated compatibility when stored at ambient conditions (about 20° C.) for up to 24 hours after admixture preparation.

Acceptable physical stability (i.e. Appearance and Description and Sub-Visible Particulates) and potency stability for piperacillin-tazobactam reconstituted with each of the following preserved diluents and then further diluted in 0.9% Sodium Chloride Injection USP. In addition, these admixtures demonstrated compatibility when stored at ambient conditions (about 20° C.) for up to 24 hours after admixture preparation.

The data also demonstrates that the product is compatible with Lactated Ringer's Injection, USP.

TABLE V
Admixture Concentrations Used To Determine Compatibility of Piperacillin Tazobactam With Various
Reconstitution and Intravenous Diluents

| Compatibility Study | | | piperacillin-tazobactam Admixture | |
|---|---|---|---|---|
| Name | Reconstitution Diluent | Admixture Diluent | Concentration | |
| Lactated Ringer's | Lactated Ringer's | Lactated Ringer's | LOW | 8 mg/mL piperacillin; |
| Preliminary Study | Injection-USP | Injection-USP | | 1 mg/mL tazobactam |
| | | | HIGH | 16 mg/mL piperacillin; |
| | | | | 2 mg/mL tazobactam |

TABLE VI
Results for Lactated Ringer's Preliminary Study

| | | USP/Ph. Eur | | | | | | | | | |
|---|---|---|---|---|---|---|---|---|---|---|---|
| Reconstitution | Piperacillin-tazobactam | Particulate Counts | | | | | | HPLC Potency Assay | | | |
| and Admixture | Admixture | 10 μm¹ | | | 25 μm² | | | Piperacillin³ | | Tazobactam³ | |
| Diluent | Concentration | t = 0 | t = 4 | t = 24 | t = 0 | t = 4 | t = 24 | t = 0 | t = 24 | t = 0 | t = 24 |
| Lactated | LOW | | | | | | | | | | |
| Ringer's | 8 mg/mL piperacillin; | 2320 | NT | 560 | 50 | NT | 0 | 100% | 101.3% | 100% | 99.6% |
| Injection-USP | 1 mg/mL tazobactam | | | | | | | | | | |
| | HIGH | | | | | | | | | | |
| | 16 mg/mL piperacillin; | 2830 | 2440 | 2100 | 80 | 110 | 80 | 100% | 98.6% | 100% | 97.9 |
| | 2 mg/mL tazobactam | | | | | | | 100%^{a} | 101.5%^{a} | 100%^{a} | 100%^{a} |

¹Not more than 6000
²Not more than 600
³Not less than 90% initial

TABLE VII
Results for Unpreserved Diluents Study

| | | USP/Ph. Eur | | | | | |
|---|---|---|---|---|---|---|---|
| Reconstitution | Piperacillin- | Particulate Counts | | | | HPLC Potency Assay | |
| and Admixture | Tazobactam Admixture | 10 μm¹ | | 25 μm² | | Piperacillin³ | Tazobactam³ |
| Diluent | Concentration | t = 0 | t = 24 | t = 0 | t = 24 | t = 24 | t = 24 |
| Lactated | LOW | 325 | 260 | 10 | 0 | | |
| Ringer's | 8 mg/mL piperacillin; | 480 | 175 | 0 | 5 | 100% | 99.4% |
| Injection-USP | 1 mg/mL tazobactam | 475 | 310 | 25 | 0 | | |
| | HIGH | 625 | 670 | 25 | 10 | | |
| | 16 mg/mL piperacillin; | 1290 | 985 | 50 | 10 | 99.7% | 100% |
| | 2 mg/mL tazobactam | 560 | 535 | 10 | 5 | | |

¹Not more than 6000
²Not more than 600
³Not less than 90% initial

TABLE VIII
Results for Reconstituted Product Hold Time Prior to Admixture Study

| | Piperacillin- | USP/Ph. Eur. | | | | | |
|---|---|---|---|---|---|---|---|
| Reconstitution | Tazobactam | Particulate Counts | | | | HPLC Potency Assay | |
| and Admixture | Admixture | 10 μm¹ | | 25 μm² | | Piperacillin³ | Tazobactam³ |
| Diluent | Concentration | t = 0 | t = 24 | t = 0 | t = 24 | t = 24 | t = 24 |
| Lactated | LOW | 950 | 975 | 20 | 40 | | |
| Ringer's | 8 mg/mL piperacillin; | 990 | 1595 | 15 | 140 | 100.66 | 101.34 |
| Injection-USP | 1 mg/mL tazobactam | 1645 | 865 | 35 | 55 | | |
| | HIGH | 1245 | 350 | 15 | 5 | | |
| | 16 mg/mL piperacillin; | 1600 | 370 | 35 | 35 | 98.79 | 100.11 |
| | 2 mg/mL tazobactam | 360 | 345 | 15 | 20 | | |

¹Not more than 6000
²Not more than 600
³Not less than 90% initial

TABLE IX
Results for European Lactated Ringer's Study from Refrigerated Concentrate

| | USP/Ph. Eur. | | | | | |
|---|---|---|---|---|---|---|
| Piperacillin-Tazobactam | Particulate Counts | | | | HPLC Potency Assay | |
| Admixture | 10 μm¹ | | 25 μm² | | Piperacillin³ | Tazobactam³ |
| Concentration | t = 0 | t = 24 | t = 0 | t = 24 | t = 24 | t = 24 |
| LOW | 547 | 1340 | 0 | 27 | | |
| 8 mg/mL piperacillin; | 520 | 377 | 3 | 17 | 96.4 | 96.5 |
| 1 mg/mL tazobactam | 447 | 910 | 23 | 70 | | |
| HIGH | 687 | 893 | 47 | 20 | | |
| 16 mg/mL piperacillin; | 1580 | 970 | 60 | 83 | 100.2 | 100.1 |
| 2 mg/mL tazobactam | 1810 | 773 | 47 | 37 | | |

¹Not more than 6000
²Not more than 600
³Not less than 90% initial

TABLE X
Results for European Lactated Ringer's 1 Week Refrigerated Study

| Piperacillin- | USP/Ph. Eur. | | | |
|---|---|---|---|---|
| Tazobactam | Particulate Counts | | HPLC Potency Assay | |
| Admixture | 10 μm¹ | 25 μm² | Piperacillin³ | Tazobactam³ |
| Concentration | t = 1 week | t = 1 week | t = 1 week | T = 1 week |
| LOW | 677 | 47 | | |
| 8 mg/mL | 367 | 13 | 95.1 | 95.7 |
| piperacillin; | | | | |
| 1 mg/mL | 667 | 3 | | |
| tazobactam | | | | |
| HIGH | 1270 | 157 | | |
| 16 mg/mL | 1433 | 37 | 98.6 | 99.1 |
| piperacillin; | | | | |
| 2 mg/mL | 1013 | 20 | | |
| tazobactam | | | | |

¹Not more than 6000
²Not more than 600
³Not less than 90% initial

What is claimed is:

1. A pharmaceutical composition which comprises piperacillin, tazobactam, an aminocarboxylic acid and a buffer in a sodium lactate diluent.

2. The pharmaceutical compositon according to claim 1, wherein the sodium lactate diluent is lactated Ringer's solution.

3. The pharmaceutical composition according to claim 1, wherein the sodium lactate diluent is Hartmann's solution.

4. The pharmaceutical composition according to claim 1 wherein the buffer is citric acid or a salt thereof.

5. The pharmaceutical composition according to claim 4, wherein the buffer is sodium citrate.

6. The pharmaceutical composition according to claim 4 in which the citrate is in the range of about 0.25 mg/ml to about 25 mg/ml.

7. The pharmaceutical composition according to claim 6 in which the citrate is in the range of about 0.6 mg/ml to about 15 mg/ml.

8. The pharmaceutical composition according to claim 1 wherein the pH is about 6.5.

9. The pharmaceutical composition according to claim 1, wherein the aminocarboxylic acid is EDTA or a salt thereof.

10. The pharmaceutical composition according to claim 9, wherein the salt of EDTA is selected from calcium disodium salt, dicalcium salt, diammonium salt, dipotassium salt, disodium salt, tetrasodium salt, tripotassium salt, and trisodium salt.

11. The pharmaceutical composition according to claim 1, wherein the aminocarboxylic acid is selected from diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N,N′,N′-tetraacetic acid (EGTA), and trans-1,2-diaminocyclohexane-N,N,N′,N′-tetraacetic acid (CyDTA) or a pharmaceutically acceptable salt thereof.

12. The pharmaceutical composition according to claim 1, in which the aminocarboxylic acid is present in the range of about 0.002 mg/ml to about 10 mg/ml.

13. The pharmaceutical composition according to claims 12, in wihich the aminocarboxylic acid is present in the range of about 0.003 to about 1 mg/ml.

14. The pharmaceutical composition according to claim 1, wherein the piperacillin is present in the range of about 8 mg/ml to about 500 mg/ml.

15. The pharmaceutical composition according to claims 14, wherein the piperacillin is present in the range of about 12 mg/ml to about 300 mg/ml.

16. The pharmaceutical composition according to claim 1, wherein the tazobactam is present in the range of about 0.1 mg/ml to about 125 mg/ml.

17. The pharmaceutical composition according to claim 16, wherein the tazobactam is present in the range of about 1.5 mg/ml to about 75 mg/ml.

18. The pharmaceutical composition according to claim 1, which further comprises an aminoglycoside.

19. The pharmaceutical composition according to claim 18, wherein the aminoglycoside is amikacin.

20. The pharmaceutical composition according to claim 18, wherein the aminoglycoside is tobramycin.

21. The pharmaceutical composition according to claim 18, in which the aminoglycoside is present in the range of about 0.1 mg/ml to about 75 mg/ml.

22. A method of treating a bacterial infection and an LR condition in a human which comprises administering to said human an effective amount of a pharmaceutical composition according to claim 1.

23. A method of treating a bacterial infection and an LR condition in a human which comprises administering to said human an effective amount of a pharmaceutical composition according to claim 18.
